SEQUENCE LISTING (11 sequences)

SEQ ID NO: 1 - DNA, 1235 bp, Homo sapiens
  CDS (27)...(1067)
ctcttccagaggcaagaccaaccaagatgagtgccttgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccccactttaacactctatgtggggaaaaagcagcttgtagagattgagaaggttgttctacaccctacctactcccaagtagatattgggctcatcaaactcaaacagaaggtgtctgttaatgagagagtgatgcccatctgcctaccatccaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgaccca

SEQ ID NO: 2 - protein, 347 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPTYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 3 - DNA, 1337 bp, Homo sapiens
  CDS (5)...(1261)
gacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgtcagctggcgcaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataaatttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacaagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctgg

SEQ ID NO: 4 - protein, 418 aa, Homo sapiens [sequence omitted]

SEQ ID NO: 5 - protein, 14 aa, Homo sapiens
LRTEGDGVYTLNDK

SEQ ID NO: 6 - protein, 11 aa, Homo sapiens
HYEGSTVPEKK

SEQ ID NO: 7 - protein, 10 aa, Homo sapiens
VTSIQDWVQK

SEQ ID NO: 8 - protein, 16 aa, Homo sapiens
DTEEEDFHVDQVTTVK

SEQ ID NO: 9 - protein, 7 aa, Homo sapiens
FLENEDR

SEQ ID NO: 10 - protein, 10 aa, Homo sapiens
LSITGTYDLK

SEQ ID NO: 11 - protein, 14 aa, Homo sapiens
LRTEGDGVYTLNEK

(19) United States
(12) Patent Application Publication
(10) Pub. No.: US 2006/0204958 A1
(43) Pub. Date: Sep. 14, 2006

(54) Methods for diagnosing htlv-i-mediated diseases

(75) Inventors: Oliver John Semmes IV, Chesapeake, VA (US); George L Wright JR., Virginia Beach, VA (US); Michael D Ward, Chesapeake, VA (US)

Correspondence Address: WILMER CUTLER PICKERING HALE AND DORR LLP, 1875 PENNSYLVANIA AVE., NW, WASHINGTON, DC, 20004, US

(73) Assignee: EASTERN VIRGINIA MEDICAL SCHOOL, NORFOLK, VA (US)

(21) Appl. No.: 10/514,476

(22) PCT Filed: May 16, 2003
(86) PCT No.: PCT/US03/15281

§ 371 Date: Jul. 11, 2005

Related U.S. Application Data
(60) Provisional application No. 60/380,854, filed on May 17, 2002.

(51) Int. Cl. C12Q 1/70; C12Q 1/68

(52) U.S. Cl. 435/6; 435/5

(57) ABSTRACT

Protein biomarkers that may advantageously be utilized in aiding in, or making, a diagnosis of HTLV-I-associated myelopathy (HAM), adult T-cell leukemia (ATL) or a negative diagnosis are described. Accordingly, in one aspect of the invention, methods for aiding in, or otherwise making, a diagnosis of ATL, HAM or a negative diagnosis are provided. Methods of detecting the protein biomarkers, kits that may be utilized to detect the biomarkers, as well as isolated protein biomarkers are also provided.

### RELATED APPLICATION

This application claims priority from U.S. Provisional Application No. 60/380,854, filed May 17, 2002, which is incorporated herein by reference.

The present invention was made with Government support under grant number CA76595 awarded by the National Institutes of Health/National Cancer Institute. The Government has certain rights in the invention.

### BACKGROUND OF THE INVENTION

The human T-cell leukemia virus type I (HTLV-I) is estimated to infect close to 20 million people worldwide as of 2002. Infection with HTLV-I can result in at least two disease states. For example, HTLV-I is the etiologic agent for adult T-cell leukemia (ATL), an aggressive lymphoproliferative disease, and HTLV-I-associated myelopathy (HAM) (also know as tropical spastic paraparesis), a chronic, progressive neurodegenerative disease clinically similar to multiple sclerosis. In endemic areas, where infection rates range from about 2% to about 30%, these diseases are major causes of mortality and morbidity (Tajima, K., Int. J. Cancer 45:237-243 (1990].

ATL is divided clinically into three groups based upon disease severity and clonality of the infected cell. The first group, smoldering leukemia, presents as a self-limiting multi-year phase typified by oligoclonal expansion/regression of T-cells. The second group, a chronic lymphoma state, is a more acute clinical entity with a polyclonal expanding T-cell population. The third group, an acute and clinically aggressive leukemia, is refractory to known treatment profiles, and marked by a monoclonal expanding T-cell population. There is some evidence that these stages may be progressive since the majority of the chronic and smoldering cases, after a relatively long period, will transform into an aggressive form [Kinoshita, K., et al., Blood 66(1):120-127 (1985); Yamano, F., et al., Cancer 55(4):851-856 (1985)].

In contrast, HAM arises from IL-2-dependent non-malignant T-cells. This cell population has been altered in ways that clearly distinguish these cells from normal T-cells. The population of HTLV-I-infected cells in a HAM patient can reach 30% of circulating peripheral blood mononuclear cells (PBMC) [Yamano, Y., et al., Blood 99(1):88-94 (2002)] These cells lack the threshold requirement for B7/CD28 [Scholz, C., et al. J. Immunol. 157(7):2932-2938 (1996)] and display an increased adherence/invasiveness perhaps explaining their ability to invade the blood/brain barrier (Romero, I. A., et al., J. Virol. 74(13):6021-6030 (2000); Trihn, D., et al. J. Biomed. Sci. 4:47-53(1996)]. The disease has been described as resulting from a complicated immunopathology, often described as a bystander cell response, which ultimately results in neurodegeneration [Izumo, S. et al., Neuropathology 20(8):Suppl. S65-S68 (2000)].

Although there are adequate methods of determining if people are infected with HTLV-I, very few diagnostic tools are available for assessing which disease state is present. For example, immunoassays are available that assay for HTLV-I gene products, HTLV-I-specific antibody production and detection of HTLV-I DNA. These parameters do not discriminate between ATL or HAM. Distinguishing or otherwise diagnosing the specific disease state at an early stage will allow treatment or other therapy to be tailored to the disease state and administered at an earlier stage to help eradicate the disease or otherwise improve the prognosis. There is a need for methods for diagnosing an HTLV-I mediated diseases state that can be performed relatively fast and inexpensively. The present invention addresses this need.

### SUMMARY OF THE INVENTION

Protein biomarkers have been discovered that may be used to diagnose, or aid in the diagnosis of, adult T-cell leukemia (ATL), HTLV-I-associated myelopathy (HAM), or to otherwise make a negative diagnosis. Accordingly, methods for aiding in, or otherwise making, a diagnosis of ATL or, HAM are provided.

In one form of the invention, a method for aiding in, or otherwise making, a diagnosis includes detecting at least one protein biomarker in a test sample from a subject. The protein biomarkers have a molecular weight selected from the group consisting of about 2488±5, about 2793±6, 2955±6, about 3965±8, about 4285±9, about 4425±8, about 4577±9, about 4913±10, about 5202±11, about 5343±11, about 5830±12, about 5874±12, about 5911±12, about 6116±12, about 6144±12, about 6366±13, about 7304±15, about 7444±15, about 8359±17, about 8609±17, about 8943±18, about 9094±18, about 9152±18, about 10113±20, about 11738±23, about 11948±24, about 12480±25, about 14706±29, and about 19900±40 Daltons. The method further includes correlating the detection with a probable diagnosis of HAM, ATL or a negative diagnosis.

In yet another aspect of the invention, methods for detecting a protein biomarker in a test sample are provided. In one form, a method may be selected from the group consisting of immunoassay and mass spectrometry. The protein biomarkers are present, absent or otherwise differentially expressed in subjects diagnosed with HAM or ATL. The protein biomarkers have a molecular weight selected from the group consisting of about 2488±5, about 2793±6, 2955±6, about 3965±8, about 4285±9, about 4425±8, about 4577±9, about 4913±10, about 5202±11, about 5343±11, about 5830±12, about 5874±12, about 5911±12, about 6116±12, about 6144±12, about 6366±13, about 7304±15, about 7444±15, about 8359±17, about 8609±17, about 8943±18, about 9094±18, about 9152±18, about 10113±20, about 11738±23, about 11948±24, about 12480±25, about 14706±29, and about 19900±40 Daltons.

In yet another aspect of the invention, kits that may be utilized to detect the biomarkers described herein and may otherwise be used to diagnose, or otherwise aid in the diagnosis of, ATL or HAM are provided. In one form of the invention, a kit includes a substrate comprising an adsorbent attached thereto, wherein the adsorbent is capable of retaining at least one protein biomarker selected from the group consisting of about 2488±5, about 2793±6, 2955±6, about 3965±8, about 4285±9, about 4425±8, about 4577±9, about 4913±10, about 5202±11, about 5343±11, about 5830 * 12, about 5874±12, about 5911±12, about 6116±12, about 6144±12, about 6366±13, about 7304±15, about 7444±15, about 8359±17, about 8609±17, about 8943±18, about 9094±18, about 9152±18, about 10113±20, about 11738±23, about 11948±24, about 12480±25, about 14706±29, and about 19900±40 Daltons; and instructions to detect the protein biomarker by contacting a test sample with the adsorbent and detecting the biomarker retained by the adsorbent.

In other aspects of the invention, isolated protein biomarkers are provided for diagnosing an HTLV-I-mediated disease state, such as HAM or ATL. In one form of the invention, the protein biomarkers have a molecular weight selected from the group consisting of about 2488±5, about 2793±6, 2955±6, about 3965±8, about 4285±9, about 4425±8, about 4577±9, about 4913±1 0, about 5202±11, about 5343±11, about 5830±12, about 5874±12, about 5911±12, about 6116±12, about 6144±12, about 6366±13, about 7304±15, about 7444±15, about 8359±17, about 8609±17, about 8943±18, about 9094±18, about 9152±18, about 10113±20, about 11738±23, about 11948±24, about 12480±25, about 14706±29, and about 19900±40 Daltons.

It is an object of the invention to provide methods to diagnose, or aid in the diagnosis of, ATL, HAM, or to otherwise make a negative diagnosis.

It is a further object of the invention to provide methods of detecting protein biomarkers in a test sample.

It is yet a further object of the invention to provide kits that may be utilized to detect the biomarkers described herein and may otherwise be used to diagnose, or otherwise aid in the diagnosis of, ATL or HAM.

It is a further object of the invention to provide isolated protein biomarkers for diagnosing an HTLV-I-mediated disease state.

These and other objects and advantages of the present invention will be apparent from the descriptions herein.

### BRIEF DESCRIPTION OF THE FIGURES

FIG. 1A depicts the high reproducibility of protein profiles processed on three different machines as discussed in Example 1. Aliquots of the pooled serum (QC) used for all quality control experiments were processed by SELDI mass spectrometry on three separate instruments. Each was a Ciphergen Protein Biomarker System version II.

FIG. 1B depicts the high reproducibility of protein profiles derived from SELDI as more fully described in Example 1. Three individual examples are shown for each class and the output was normalized to total ion current. The vertical scale deflection represents relative amount of detected protein. The horizontal axis designates the mass. ATL, adult T-cell leukemia; HAM, HTLV-associated myelopathy; normal, normal control.

FIG. 2 depicts SELDI spectra of class-specific peaks as described in Examples 1 and 2. A peak unique to ATL (4577, arrow) and absent in ATL (3965, arrow) are shown. The classes are ATL (A), HAM (H) and Normal (N).

FIG. 3 depicts SELDI data as a gel-view as described in Examples 1 and 2. A peak absent in ATL (5345) and a peak expressed at different levels in all classes (11,738) is shown.

FIG. 4 depicts differential SELDI spectra for an ATL specific protein as described in Examples 1 and 2. ATL, adult T-cell leukemia; HAM, HTLV-associated myelopathy; normal, normal control.

FIG. 5A depicts a decision tree graph for distinguishing ATL from normal controls. The initial primary splitter (11.7 kD) is shown in the circle. Each of the subsequent splitters are shown in boxes. The terminal nodes are indicated as end-of-path boxes. The training with 5 fold cross validation resulted in 94% sensitivity and 97% specificity.

FIG. 5B shows a scatter plot diagram for the primary and secondary tree decision nodes of FIG. 5A. The scatter plot depicts the relative variation in expression values for each decision event for the training set. The decision cut-off is represented by a horizontal line; samples are referred to the secondary node which is chosen based upon the value displayed.

FIG. 6A shows a decision tree for distinguishing ATL from HAM and Normal as more fully described in Example 2. The primary decision splitter is shown in the circle (node 1). The secondary splitter is shown in a box (node 2) and the terminal decision bins are shown as end branch squares (terminal node 1, 2 and 3).

FIG. 6B depicts a decision tree for distinguishing HAM from normals as more fully described in Example 2. The primary decision splitter is in the circle (node 1). The secondary decision splitters are in squares (node 2 and 3) and the terminal decision bins are shown as terminal squares (Terminal node 1, 2, and 3).

FIG. 6C shows decision tree resulting from combining the trees of FIGS. 6A and 6B as more fully described in Example 2. The trees developed for separating ATL from HAM+Normal and separating HAM from Normal were used in tandem to classify a separate test data set. The full test set (10 ATL, 10 HAM, 10 Normal) enters the tree as shown at the primary decision node (First Split). Arrows depict the path of samples following the decisions. Terminal bins are shown at dead-end nodes and the samples indicated. Miss-classified samples in terminal bins are shown by an asterisk (*). The sequence followed is First Split, Second Split, Third Split, Fourth Split and Fifth Split.

FIG. 7 depicts an expression profile and retentate map of the region surrounding the 11.7 kD peak (arrow). The upper panel is the TOF expression profile and the lower panel is the retentate map depiction of the same data. Shown are the pairs of each patient type as displayed; adult t-cell leukemia (ATL-1, ATL-2), HTLV-1-associated myelopathy (HAM-1, HAM-2) and normal (Nor-1, Nor-2).

FIG. 8 depicts an SDS polyacrylamide gel showing a 12 kD protein band. ATL (A) and normal (N) serum pairs were reacted with an IMAC Cu²⁺ column and the bound proteins were eluted, loaded on an SDS polyacrylamide gel and stained with Fast silver, all as described in Example 2.

FIG. 9 depicts peptide identities identified by mass spectrometry/mass spectrometry (MS/MS) as more fully discussed in Example 2. The position of three peptides (A, B and C) found within the 11.7 kD region of human alpha-1-antitrypsin, along with the sequence of each peptide, are depicted. The A peptide was found 2 times. Further shown are the position of peptides A and B within the 19.9 kD fragment, and the position of peptides C and D within the 11.9 kD fragment, of human haptoglobin-2. The B peptide was found seven times. Each of the regions identified within human haptoglobin-2 were achieved with two separate peptides. Shown for each peptide identified is the cross-correlation (Xcorr), delta-correlation (dCn) and the ion spread (Ions).

### DESCRIPTION OF THE PREFERRED EMBODIMENTS

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

The present invention relates to methods for aiding in a diagnosis of, and methods for diagnosing, HTLV-I-mediated diseases, including HTLV-I-associated myelopathy (HAM) [also known as tropical spastic paraparesis (TSP)] and adult T cell leukemia (ATL). The method offers a rapid and simple approach to the determination of disease lineage and for the predication of disease outcome utilizing easily obtainable test samples, such as those from biological fluids, including blood and blood sera. In preferred forms of the invention, a diagnosis may be made by analyzing no more than about 50 μl of blood. The method takes advantage of the discovery of protein biomarkers whose presence, absence and/or quantity or otherwise differential expression in the aforementioned diseases states may be correlated to the specific disease state. Accordingly, such protein biomarkers are also provided herein. Methods for detecting the biomarkers are also described herein, as are kits for aiding in, or for otherwise making, a diagnosis of ATL, HAM or a negative diagnosis.

In one aspect of the invention, methods for diagnosing HTLV-I-mediated diseases, such as ATL or HAM, are provided. In one form, a method includes detecting at least one protein biomarker in a test sample. The protein biomarker typically has a molecular weight of about 20,000 Daltons or less, and in preferred forms of the invention may be selected from protein biomarkers having a molecular weight of about 2488±5, about 2793±6, 2955±6, about 3965±8, about 4285±9, about 4425±8, about 4577±9, about 4913±10, about 5202±11, about 5343±11, about 5830±12, about 5874±12, about 5911±12, about 6116±12, about 6144±12, about 6366±13, about 7304±15, about 7444±15, about 8359±17, about 8609±17, about 8943±18, about 9094±18, about 9152±18, about 10113±20, about 11738±23, about 11948±24, about 12480±25, about 14706±29, and about 19900±40 Daltons. In further preferred forms of the invention, the protein biomarkers have a molecular weight of about 3965±8, about 4425±8, about 4577±9, about 5345±11, about 8359±17, about 11738±23, and about 19900±40 Daltons. The detection may then be correlated to a diagnosis of HAM, ATL or normal (i.e., not diagnosed as having HAM or ATL) or an otherwise negative diagnosis. As used herein, the term “detecting” includes determining the presence, the absence, the quantity, or a combination thereof, of the protein biomarkers.

In one form of the invention, the method may be used to diagnose, or aid in the diagnosis of, ATL by detecting, for example, the presence or absence of ATL-specific biomarkers. For example, the presence of at least one of the about 2488±5, the about 5202±11, the about 7304±15, the about 12480±25 and the about 19900±40 Dalton biomarkers may be correlated to a probable diagnosis of ATL. Moreover, the absence of protein biomarkers having a molecular weight of about 3965±8, about 5830±12, about 6366±13, about 8359±17, and about 9152±18 Daltons may be correlated to a probable diagnosis of ATL. Additionally, either the absence, or the differential expression as described below, of the about 5345±11 Dalton biomarker may be correlated to a probable diagnosis of ATL.

In another embodiment, the method may be used to diagnose, or aid in the diagnosis of, HAM by detecting, for example, the presence of HAM-specific protein biomarkers. For example, the presence in a test sample from a subject of at least one protein biomarker having a molecular weight of about 4913±10, about 6144±12, and about 7444±15 Daltons may be correlated to a probable diagnosis of HAM.

In other embodiments of the invention, the differential expression, such as the over- or under-expression, of selected protein biomarkers may be correlated to a particular disease state. By differentially expressed, it is meant herein that the protein biomarkers may be found at a greater or smaller level in one disease state compared to another, or that it may be found at a higher frequency in one or more disease states. In one form of the invention, selected protein biomarkers in test samples from subjects with ATL may be elevated compared to normal individuals. For example, the about 2793±6 Dalton biomarker is present about 2-fold more, the about 4425±8 Dalton biomarker is present about 7-fold more, the about 4577±9 Dalton biomarker is present about 23-fold more, the about 5874±11 Dalton biomarker is present about 3-fold more, the about 9094±18 Dalton biomarker is present about 4-fold more, the about 10113±20 Dalton biomarker is present about 3-fold more, the about 11738±23 Dalton biomarker is present about 3-fold more, the about 11948±24 Dalton biomarker is present about 2-fold more, the about 13369±27 Dalton biomarker is present about 2-fold more, the about 14706±29 Dalton biomarker is present about 2-fold more, and the about 19900±40 is present about 4-fold more in test samples from individuals with ATL compared to normal individuals. In another form of the invention, selected biomarkers in test subjects with ATL may be decreased compared to normal individuals. For example, the about 4290±9 Dalton biomarker is about 3-fold lower, the about 5345±11 Dalton biomarker is about 10-fold lower, and the about 5914±12 Dalton biomarker is about 4-fold lower in test samples from individuals with ATL compared to normal individuals.

The over- or under-expression of selected biomarkers may also be correlated to a diagnosis of HAM or a negative diagnosis. For example, the about 4577±9 Dalton biomarker is present about 3-fold more, and the about 8613±17 Dalton, and the about 19900±40 Dalton biomarkers are present about 2-fold more in test samples from individuals with HAM compared to normal individuals.

It can thus be seen that analyzing a test sample for the presence, absence or quantity of at least one protein biomarker will aid in a diagnosis, or in making a diagnosis, of ATL, HAM or in making a negative diagnosis. Although a single biomarker may be utilized, it is preferred that two, three, four, five, six, seven, eight, nine or more, such as all twenty-nine, of the biomarkers are analyzed, with respect to some combination of its presence, absence or quantity, to make a diagnosis. Thus, not only can one or more protein biomarkers be detected, one to six, one to nine, one to twenty-nine, or some combination, may be detected and analyzed as described herein. In addition, other protein biomarkers not herein described may be combined with any of the presently disclosed protein biomarkers to aid in making, or otherwise make, a diagnosis of ATL, HAM or a negative diagnosis.

The test sample may be obtained from a wide variety of sources. The sample is typically obtained from biological fluid from a subject or patient who is being tested for ATL or HAM, who is thought to be at risk for ATL or HAM, who is thought to have ATL or HAM or any test subject in which it is desired to diagnose ATL or HAM. A preferred biological fluid is blood or blood sera. Other biological fluids in which the biomarkers may be found include, for example, saliva, tears, lymph fluid, sputum, mucus, lung/bronchial washes, urine, or other similar fluid. Additionally, the test samples may be obtained, for example, from animals, such as mammals and preferably from humans.

The detection of the protein biomarkers described herein may be performed in a variety of ways. Accordingly, methods for detecting a protein biomarker in a test sample are provided herein.

In one form of the invention, a method for detecting the biomarker includes detecting the biomarker by gas phase ion spectrometry utilizing a gas phase ion spectrometer. The method may include contacting a test sample having a biomarker, such as the protein biomarkers described herein, with a substrate comprising an adsorbent thereon under conditions to allow binding between the biomarker and the adsorbent and detecting the biomarker bound to the adsorbent by gas phase ion spectrometry.

A wide variety of adsorbents may be used. The adsorbents include a hydrophobic group, a hydrophilic group, a cationic group, an anionic group, a metal ion chelating group, or antibodies which specifically bind to an antigenic biomarker, or some combination thereof (such as a “mixed mode” adsorbent). Exemplary adsorbents that include a hydrophobic group include matrices having aliphatic hydrocarbons, such as C₁-C₁₈ aliphatic hydrocarbons and matrices having aromatic hydrocarbon functional groups, including phenyl groups. Exemplary adsorbents that include a hydrophilic group include silicon oxide, or hydrophilic polymers such as polyalkylene glycol, such as polyethylene glycol; dextran, agarose or cellulose. Exemplary adsorbents that include a cationic group include matrices of secondary, tertiary or quaternary amines. Exemplary adsorbents that have an anionic group include matrices of sulfate anions and matrices of carboxylate anions or phosphate anions. Exemplary adsorbents that have metal chelating groups include organic molecules that have one or more electron donor groups which may form coordinate covalent bonds with metal ions, such as copper, nickel, cobalt, zinc, iron, aluminum and calcium. Exemplary adsorbents that include an antibody include antibodies that are specific for any of the biomarkers provided herein and may be readily made by methods known to the skilled artisan.

In a further form, the substrate can be in the form of a probe, which may be removably insertable into a gas phase ion spectrometer. For example, a substrate may be in the form of a strip with adsorbents on its surface. In yet other forms of the invention, the substrate can be positioned onto a second substrate to form a probe which may be removably insertable into a gas phase ion spectrometer. For example, the substrate can be in the form of a solid phase, such as a polymeric or glass bead with a functional group for binding the marker, which can be positioned on a second substrate to form a probe. The second substrate may be in the form of a strip, or a plate having a series of wells at predetermined locations. In this form of the invention, the marker can be adsorbed to the first substrate and transferred to the second substrate which can then be submitted for analysis by gas phase ion spectrometry.

The probe can be in the form of a wide variety of desired shapes, including circular, elliptical, square, rectangular, or other polygonal or other desired shape, as long as it is removably insertable into a gas phase ion spectrometer. The probe is also preferably adapted or otherwise configured for use with inlet systems and detectors of a gas phase ion spectrometer. For example, the probe can be adapted for mounting in a horizontally and/or vertically translatable carriage that horizontally and/or vertically moves the probe to a successive position without requiring, for example, manual repositioning of the probe.

The substrate that forms the probe can be made from a wide variety of materials that can support various adsorbents. Exemplary materials include insulating materials, such as glass and ceramic; semi-insulating materials, such as silicon wafers; electrically-conducting materials (including metals such as nickel, brass, steel, aluminum, gold or electrically-conductive polymers); organic polymers; biopolymers, or combinations thereof.

In other embodiments of the invention, depending on the nature of the substrate, the substrate surface may form the adsorbent. In other cases, the substrate surface may be modified to incorporate thereon a desired adsorbent. The surface of the substrate forming the probe can be treated or otherwise conditioned to bind adsorbents that may bind markers if the substrate can not bind biomarkers by itself. Alternatively, the surface of the substrate can also be treated or otherwise conditioned to increase its natural ability to bind desired biomarkers. Other probes suitable for use in the invention may be found, for example, in PCT International Publication Nos. WO 01/25791 (Tai-Tung et al.) and WO 01/71360 (Wright et al.).

The adsorbents may be placed on the probe substrate in a wide variety of patterns, including a continuous or discontinuous pattern. A single type of adsorbent, or more than one type of adsorbent, may be placed on the substrate surface. The patterns may be in the form of lines, curves, such as circles, or other shape or pattern as desired and as known in the art.

The method of production of the probes will depend on the selection of substrate materials and/or adsorbents as known in the art. For example, if the substrate is a metal, the surface may be prepared depending on the adsorbent to be applied thereon. For example, the substrate surface may be coated with a material, such as silicon oxide, titanium oxide or gold, that allows derivatization of the metal surface to form the adsorbent. The substrate surface may then be derivatized with a bifunctional linker, one of which binds, such as covalently binds, with a functional group on the surface and the opposing end of the linker may be further derivatized with groups that function as an adsorbent. As a further example, a substrate that includes a porous silicon surface generated from crystalline silicon can be chemically modified to include adsorbents for binding markers. Additionally, adsorbents with a hydrogel backbone can be formed directly on the substrate surface by in situ polymerization of a monomer solution which includes, for example, substituted acrylamide or acrylate monomers, or derivatives thereof that include a functional group of choice as adsorbent.

In preferred forms of the invention, the probe may be a chip, such as those available from Ciphergen Biosystems, Inc. (Palo Alto, Calif.). The chip may be a hydrophilic, hydrophobic, anion-exchange, cation-exchange, immobilized metal affinity or preactivated protein chip array. The hydrophobic chip may be a ProteinChip H4, which includes a long-chain aliphatic surface that binds proteins by reverse phase interaction. The hydrophilic chip may be ProteinChips NP1 and NP2 which include a silicon dioxide substrate surface. The cation exchange Proteinchip array may be Proteinchip WCX2, a weak cation exchange array with a carboxylate surface to bind cationic proteins. Alternatively, the chip may be an anion exchange protein chip array, such as SAX1 (strong anion exchange) ProteinChip which are made from silicon-dioxide-coated aluminum substrates, or ProteinChip SAX2 with a higher capacity quaternary ammonium surface to bind anionic proteins. A further useful chip may be the immobilized metal affinity capture chip (IMAC3) having nitrilotriacetic acid on the surface. Further alternatively, ProteinChip PS1 is available which includes a carbonyldiimidazole surface which covalently reacts with amino groups or may be ProteinChip PS2 which includes an epoxy surface which covalently reacts with amine and thiol groups.

In one form of a method of detection of a biomarker, the probe contacts a test sample. The test sample is preferably a biological fluid sample as previously described herein. In a preferred form of the invention, the sample is a blood serum sample. If necessary, the sample can be solubilized in or mixed with an eluant prior to being contacted with the probe. The probe may contact the test sample solution by a wide variety of techniques, including bathing, soaking, dipping, spraying, washing, pipetting or other desirable methods. The method is performed so that the adsorbent of the probe preferably contacts the test sample solution. Although the concentration of the biomarker or biomarkers in the sample may vary, it is generally desirable to contact a volume of test sample that includes about 1 attomole to about 100 picomoles of marker in about 1 μl to about 500 μl solution for binding to the adsorbent.

The sample and probe contact each other for a period of time sufficient to allow the biomarker to bind to the adsorbent. Although this time may vary depending on the nature of the sample, the nature of the biomarker, the nature of the adsorbent and the nature of the solution the biomarker is dissolved in, the sample and adsorbent are typically contacted for a period of about 30 seconds to about 12 hours, preferably about 30 seconds to about 15 minutes.

The temperature at which the probe contacts the sample will depend on the nature of the sample, the nature of the biomarker, the nature of the adsorbent and the nature of the solution the biomarker is dissolved in. Generally, the sample may be contacted with the probe under ambient temperature and pressure and conditions. However, the temperature and pressure may vary as desired. For example, the temperature may vary from about 4° C. to about 37° C.

After the sample has contacted the probe for a period of time sufficient for the marker to bind to the adsorbent or substrate surface should no adsorbent be used, unbound material may be washed from the substrate or adsorbent surface so that only bound materials remain on the respective surface. The washing can be accomplished by, for example, bathing, soaking, dipping, rinsing, spraying or otherwise washing the respective surface with an eluant or other washing solution. A microfluidics process is preferably used when a washing solution such as an eluant is introduced to small spots of adsorbents on the probe. The temperature of the washing solution may vary, but is typically about 0° C. to about 100° C., and preferably about 4° C. and about 37° C.

A wide variety of washing solutions may be utilized to wash the probe substrate surface. The washing solutions may be organic solutions or aqueous solutions. Exemplary aqueous solutions may be buffered solutions, including HEPES buffer, a Tris buffer, phosphate buffered saline or other similar buffers known to the art. The selection of a particular washing solution will depend on the nature of the biomarkers and the nature of the adsorbent utilized. For example, if the probe includes a hydrophobic group and a sulfonate group as adsorbents, such as the SCXI PorteinChip® array, then an aqueous solution, such as a HEPES buffer, may be used. As a further example, if a probe includes a metal binding group as an adsorbent, such as with the Ni(II) ProteinChip® array, than an aqueous solution, such as a phosphate buffered saline may be preferred. As yet a further example, if a probe include a hydrophobic group as an adsorbent, such as with the HF ProteinChip® array, water may be a preferred washing solution.

An energy absorbing molecule, such as one in solution, may be applied to the markers or other substances bound on the substrate surface of the probe. As used herein, an “energy absorbing molecule” refers to a molecule that absorbs energy from an energy source in a gas phase ion spectrometer, which may assist the desorption of markers or other substances from the surface of the probe. Exemplary energy absorbing molecules include cinnamic acid derivatives, sinapinic acid, dihyroxybenzoic acid and other similar molecules known to the art. The energy absorbing molecule may be applied by a wide variety of techniques previously discussed herein for contacting the sample and probe substrate, including, for example, spraying, pipetting or dipping, preferably after the unbound materials are washed off the probe substrate surface.

After the marker is appropriately bound to the probe, it may be detected, quantified and/or its characteristics may be otherwise determined using a gas phase ion spectrometer. As known in the art, gas phase ion spectrometers include, for example, mass spectrometers, ion mobility spectrometers, and total ion current measuring devices.

In a preferred embodiment, a mass spectrometer is utilized to detect the biomarkers bound to the substrate surface of the probe. The probe with the bound marker on its surface, may be introduced into an inlet system of the mass spectrometer. The marker may then be ionized by an ionization source, such as a laser, fast atom bombardment, plasma or other suitable ionization sources known to the art. The generated ions are typically collected by an ion optic assembly and a mass analyzer then disperses and analyzes the passing ions. The ions exiting the mass analyzer are detected by a detector. The detector translates information of the detected ions into mass-to-charge ratios. Detection and/or quantitation of the marker will typically involve detection of signal intensity.

In further preferred forms of the invention, the mass spectrometer is a laser desorption time-of-flight mass spectrometer, and further preferably surface enhanced laser desorption time-of-flight mass spectrometry (SELDI-TOF-MS) is utilized. SELDI is an improved method of gas phase ion spectrometry for biomolecules. In SELDI, the surface on which the analyte is applied plays an active role in the analyte capture, and/or desorption.

As known in the art, in laser desorption mass spectrometry, a probe with a bound marker is introduced into an inlet system. The marker is desorbed and ionized into the gas phase by a laser ionization source. The ions generated are collected by an ion optic assembly. Ions are accelerated in a time-of-flight mass analyzer through a relatively short high voltage field and allowed to drift into a high vacuum chamber. The accelerated ions strike a sensitive detector surface at a far end of the high vacuum chamber at different times, which are characteristic for a given ion and reproducible. As the time-of-flight is a function of the mass of the ions, the elapsed time between ionization and impact can be used to identify the presence or absence of molecules of specific mass. Quantitation of the biomarkers, either in relative or absolute amounts, may be accomplished by comparison of the intensity of the displayed signal of the biomarker to a control amount of a biomarker or other standard as known in the art. The components of the laser desorption time-of-flight mass spectrometer may be combined with other components described herein and/or known to the skilled artisan that employ various means of desorption, acceleration, detection, or measurement of time.

In further embodiments, detection and/or quantitation of the biomarkers may be accomplished by matrix-assisted laser desorption ionization (MALDI). MALDI also provides for vaporization and ionization of biological samples from a solid-state phase directly into the gas phase. As known in the art, the sample including the desired analyte is dissolved in, or otherwise suspended in, a matrix that co-crystallizes with the analyte, preferably to prevent the degradation of the analyte during the process.

In another form of the invention, an ion mobility spectrometer can be used to detect and characterize the biomarkers described herein. The principle of ion mobility spectrometry is based on the different mobilities of ions. Specifically, ions of a sample produced by ionization move at different rates, due to their difference in, for example, mass, charge, or shape, through a tube under the influence of an electric field. The ions (typically in the form of a current) are registered at the detector which can then be used to identify a marker or other substances in the sample. One advantage of ion mobility spectrometry is that it can operate at atmospheric pressure.

In another embodiment, a total ion current measuring device can be used to detect and characterize the biomarkers described herein. This device can be used, for example, when the probe has a surface chemistry that allows only a single type of marker to be bound. When a single type of marker is bound on the probe, the total current generated from the ionized biomarker reflects the nature of the marker. The total ion current produced by the biomarker can then be compared to stored total ion current of known compounds. Characteristics of the biomarker can then be determined.

Data generated by desorption and detection of the biomarkers can be analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. Using this information, the program can then identify the set of features on the probe defining certain selectivity characteristics, such as types of adsorbents and eluants used. The computer also contains code that receives data on the strength of the signal at various molecular masses received from a particular addressable location on the probe as input. This data can indicate the number of biomarkers detected, optionally including the strength of the signal and the determined molecular mass for each biomarker detected.

Data analysis can include the steps of determining signal strength (e.g., height of peaks, area of peaks) of a biomarker detected and removing “outerliers” (data deviating from a predetermined statistical distribution). For example, the observed peaks can be normalized, a process whereby the height of each peak relative to some reference is calculated. For example, a reference can be background noise generated by instrument and chemicals (e.g., energy absorbing molecule) which is set as zero in the scale. The signal strength can then be detected for each biomarker or other substances can be displayed in the form of relative intensities in the scale desired (e.g., 100). Alternatively, a standard may be included with the sample so that a peak from the standard can be used as a reference to calculate relative intensities of the signals observed for each biomarker or other markers detected.

The computer can transform the resulting data into various formats for displaying. In one exemplary format, referred to as “spectrum view or retentate map,” a standard spectral view can be displayed, wherein the view depicts the quantity of biomarker reaching the detector at each particular molecular weight. In another exemplary format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In a further exemplary format, referred to as “3-D overlays,” several spectra can be overlayed to study subtle changes in relative peak heights. In yet a further exemplary format, referred to as “difference map view,” two or more spectra can be compared, conveniently highlighting unique biomarkers and biomarkers which are up- or down-regulated between samples. Biomarker profiles (spectra) from any two samples may be compared visually.

Using any of the above display formats, it can be readily determined from the signal display whether a biomarker having a particular molecular weight is detected from a sample. Moreover, from the strength of signals, the amount of markers bound on the probe surface can be determined.

The test samples may be pre-treated prior to being subject to gas phase ion spectrometry. For example, the samples can be purified or otherwise pre-fractionated to provide a less complex sample for analysis. The optional purification procedure for the biomolecules present in the test sample may be based on the properties of the biomolecules, such a size, charge and function. Methods of purification include centrifugation, electrophoresis, chromatography, dialysis or a combination thereof. As known in the art, electrophoresis may be utilized to separate the biomolecules in the sample based on size and charge. Electrophoretic procedures are well known to the skilled artisan, and include isoelectric focusing, sodium dodecyl sulfate polyacrylamide gel electrophoresis (SDS-PAGE), agarose gel electrophoresis, and other known methods of electrophoresis.

The purification step may be accomplished by a chromatographic fractionation technique, including size fractionation, fractionation by charge and fractionation by other properties of the biomolecules being separated. As known in the art, chromatographic systems include a stationary phase and a mobile phase, and the separation is based upon the interaction of the biomolecules to be separated with the different phases. In preferred forms of the invention, column chromatographic procedures may be utilized. Such procedures include partition chromatography, adsorption chromatography, size-exclusion chromatography, ion-exchange chromatography and affinity chromatography. Such methods are well known to the skilled artisan. In size exclusion chromatography, it is preferred that the size fractionation columns exclude molecules whose molecular mass is greater than about 20,000 Daltons.

In a preferred form of the invention, the sample is purified or otherwise fractionated on a bio-chromatographic chip by retentate chromatography before gas phase ion spectrometry. A preferred chip is the Protein Chip™ available from Ciphergen Biosystems, Inc. (Palo Alto, Calif.). As described above, the chip or probe is adapted for use in a mass spectrometer. The chip comprises an adsorbent attached to its surface. This adsorbent can function, in certain applications, as an in situ chromatography resin. In operation, the sample is applied to the adsorbent in an eluant solution. Molecules for which the adsorbent has affinity under the wash condition bind to the adsorbent. Molecules that do not bind to the adsorbent are removed with the wash. The adsorbent can be further washed under various levels of stringency so that analytes are retained or eluted to an appropriate level for analysis. An energy absorbing molecule can then be added to the adsorbent spot to further facilitate desorption and ionization. The analyte is detected by desorption from the adsorbent, ionization and direct detection by a detector. Thus, retentate chromatography differs from traditional chromatography in that the analyte retained by the affinity material is detected, whereas in traditional chromatography, material that is eluted from the affinity material is detected.

In yet another form of the invention, the biomarkers of the present invention may be detected, qualitatively or quantitatively, by an immunoassay procedure. The immunoassay typically includes contacting a test sample with an antibody that specifically binds to or otherwise recognizes a biomarker, and detecting the presence of a complex of the antibody bound to the biomarker in the sample. The biomarker is preferably one that is present, absent or differentially expressed in subjects diagnosed with an HTLV-I-mediated disease selected from HAM or ATL. Additionally, the biomarkers preferably have a molecular weight selected from the group consisting of about 2488±5, about 2793±6, 2955±6, about 3965±8, about 4285±9, about 4425±8, about 4577±9, about 4913±10, about 5202±11, about 5343±11, about 5830±12, about 5874±12, about 5911±12, about 6116±12, about 6144±12, about 6366±13, about 7304±15, about 7444±15, about 8359±17, about 8609±17, about 8943±18, about 9094±18, about 9152±18, about 10113±20, about 11738±23, about 11948±24, about 12480±25, about 14706±29, and about 19900±40 Daltons. In preferred forms of the invention, the protein biomarkers have a molecular weight selected from the group consisting of about 3965±8, about 4425±8, about 4577±9, about 5343±11, about 8359±17, about 11738±23, and about 19900±40 Daltons. The immunoassay procedure may be selected from a wide variety of immunoassay procedures known to the art involving recognition of antibody/antigen complexes, including enzyme immunoassays, competitive or non-competitive, and including enzyme-linked immunosorbent assays (ELISA), radioimmunoassays (RIA) and Western blots. Such assays are well known to the skilled artisan and are described, for example, more thoroughly in Antibodies: A Laboratory Manual (1988) by Harlow & Lane; Immunoassays: A Practical Approach, Oxford University Press, Gosling, J. P. (ed.) (2001) and/or Current Protocols in Molecular Biology (Ausubel et al.) which is regularly and periodically updated.

The antibodies to be used in the immunoassays described herein may be polyclonal antibodies and may be obtained by procedures which are well known to the skilled artisan, including injecting isolated, or otherwise purified biomarkers into various animals and isolating the antibodies produced in the blood serum. The antibodies may be monoclonal antibodies whose method of production is well known to the art, including injecting isolated, or otherwise purified biomarkers into a mouse, for example, isolating the spleen cells producing the anti-serum, fusing the cells with tumor cells to form hybridomas and screening the hybridomas. The biomarkers may first be purified by techniques similarly well known to the skilled artisan, including the chromatographic, electrophoretic and centrifugation techniques described previously herein. Such procedures may take advantage of the protein biomarker's size, charge, solubility, affinity for binding to selected components, combinations thereof, or other characteristics or properties of the protein. Such methods are known to the art and can be found, for example, in Current Protocols in Protein Science, J. Wiley and Sons, New York, N.Y., Coligan et al. (Eds.) (2002; Harris, E. L. V., and S. Angal in Protein purification applications: a practical approach, Oxford University Press, New York, N.Y. (1990). Once the antibody is provided, a biomarker can be detected and/or quantitated by the immunoassays previously described herein.

Although specific procedures for immunoassays are well known to the skilled artisan, an immunoassay may be performed by initially obtaining a sample as previously described herein from a test subject. The antibody may be fixed to a solid support prior to contacting the antibody with a test sample to facilitate washing and subsequent isolation of the antibody/protein biomarker complex. Examples of solid supports are well known to the skilled artisan and include, for example, glass or plastic in the form of, for example, a microtiter plate. Antibodies can also be attached to the probe substrate, such as the ProteinChip™ arrays described herein.

After incubating the test sample with the antibody, the mixture is washed and the antibody-marker complex may be detected. The detection can be accomplished by incubating the washed mixture with a detection reagent, and observing, for example, development of a color or other indicator. The detection reagent may be, for example, a second antibody which is labeled with a detectable label. Exemplary detectable labels include magnetic beads (e.g., DYNABEADS™), fluorescent dyes, radiolabels, enzymes (e.g., horseradish peroxide, alkaline phosphatase and others commonly used in enzyme immunoassay procedures), and colorimetric labels such as colloidal gold, colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture. The amount of an antibody-marker complex can be determined by comparing to a standard.

Throughout the assays, incubation and/or washing steps may be required after each combination of reagents. Incubation steps can vary from about 5 seconds to several hours, preferably from about 5 minutes to about 24 hours. However, the incubation time will depend upon the particular immunoassay, biomarker, and assay conditions. Usually the assays will be carried out at ambient temperature, although they can be conducted over a range of temperatures, such as about 0° C. to about 40° C.

In yet another aspect of the invention, kits are provided that may, for example, be utilized to detect the biomarkers described herein. The kits can, for example, be used to detect any one or more of the biomarkers described herein which may advantageously be utilized for diagnosing, or aiding in the diagnosis of, HAM, ATL or in a negative diagnosis. In one embodiment, a kit may include a substrate that includes an adsorbent thereon, wherein the adsorbent is preferably suitable for binding one or more protein biomarkers described herein, and instructions to detect the biomarker by contacting a test sample as described herein with the adsorbent and detecting the biomarker retained by the adsorbent. In certain embodiments, the kits may include an eluant, or instructions for making an eluant, wherein the combination of the eluant and the adsorbent allows detection of the protein biomarkers by, for example, use of gas phase ion spectrometry. Such kits can be prepared from the materials described herein. In yet another embodiment, the kit may include a first substrate that includes an adsorbent thereon (e.g., a particle functionalized with an adsorbent) and a second substrate onto which the first substrate can be positioned to form a probe which is removably insertable into a gas phase ion spectrometer. In other embodiments,,the kit may include a single substrate which is in the form of a removably insertable probe with adsorbents on the substrate. In yet another embodiment, the kit may further include a pre-fractionation spin column (e.g, K-30 size exclusion column).

The kit may further include instructions for suitable operating parameters in the form of a label or a separate insert. For example, the kit may have standard instructions informing a consumer or other individual how to wash the probe after a particular form of sample is contacted with the probe. As a further example, the kit may include instructions for pre-fractionating a sample to reduce the complexity of proteins in the sample.

In a further embodiment, a kit may include an antibody that specifically binds to the marker and a detection reagent. Such kits can be prepared from the materials described herein. The kit may further include pre-fractionation spin columns as described above, as well as instructions for suitable operating parameters in the form of a label or a separate insert.

In yet another aspect of the invention, isolated or otherwise purified, biomarkers for diagnosing an HTLV-I-mediated disease state are provided. The term “isolated protein biomarker” as used herein is intended to refer to a protein biomarker which is not in its native environment. For example, the protein is separated from contaminants that naturally accompany it such as lipids, nucleic acids, carbohydrates and other proteins. The term includes proteins which have been removed or purified from their naturally occurring environment and further includes protein isolates and chemically synthesized proteins. In one form of the invention, the protein biomarkers are present, absent and/or differentially expressed in subjects diagnosed with an HTLV-I mediated disease state, such as HAM or ATL. The proteins typically have molecular weights of about 20,000 Da or less. In preferred forms of the invention, the protein biomarkers have molecular weights selected from the group consisting of about 2488±5, about 2793±6, 2955±6, about 3965±8, about 4285±9, about 4425±8, about 4577±9, about 4913±10, about 5202±11, about 5343±11, about 5830±12, about 5874±12, about 5911±12, about 6144±12, about 6116±12, about 6366±13, about 7304±15, about 7444±15, about 8359±17, about 8609±17, about 8943±18, about 9094±18, about 9152±18, about 10113±20, about 11738±23, about 11948±24, about 12480±25, about 14706±29, and about 19900±40 Daltons. Additionally, the proteins are metal-binding proteins. As defined herein, the term “metal-binding proteins” are proteins that have an affinity for binding to metals, including metal ions such as, for example, Cu²⁺, Zn²⁺, Ni²⁺ and Mg²⁺.

Reference will now be made to specific examples illustrating the compositions and methods above. It is to be understood that the examples are provided to illustrate preferred embodiments and that no limitation to the scope of the invention is intended thereby.

### EXAMPLE 1

### Application of SELDI-TOF-Mass Spectrometry to sera Protein Profiling of HTLV-I-Infected Patients

Materials and Methods

Sample Acquisition and Preparation

Whole blood was drawn from individuals following consent. The blood was collected in a 10 cc Serum Separator Vacutainer Tube and centrifuged for 5 minutes at 3750 rpm to separate out the serum fraction. Serum was immediately transferred to ice. The samples were then aliquoted into 500 μl fractions and stored at −70° C. following no more than a 6 hour delay. Each fraction was limited to a single freeze-thaw prior to analysis.

SELDI-TOF-MS/Classification Algorithm

The algorithm used in the Classification Logic is based on cumulative probability. Essentially, a separate profile is generated for the expression data and the presence/absence (P/A) data that takes into account each cluster's Overall Incidence (how often the peak appears in the whole sample population), the group incidence (what % of the samples in a particular group have that peak), and for expression data, the coefficient of variation (CV %) of the intensity. In addition, for the P/A data, a function was written that calculates the degree of variability of a cluster, so that the incidence for a peak m/z=5000 is ATL (100%), HAM(2%), Normal(0%) would be more weighted for ATL than m/z=2000 at ATL(50%), HAM(10%), Normal(5%).

Results

Group 1

Protein profiling as described herein has revealed signature expression “fingerprints” specific for ATL and HAM. In this example, a group of serum specimens from the NIH was used. The group was pre-classified as ATL (n=15), HAM (n=30) and control (n=30). There was no front-end selection for clinical severity and the controls were not known to be HTLV-I-infected.

Reproducibility of Spectral Data

A key aspect of any clinical approach for reliable disease diagnostics and early detection is reproducibility. Several steps have been developed that are essential for reproducibility in the SELDI process. Optimal performance parameters have been established beyond the standard calibration steps that enable initialization and monitoring of the performance of the instrument. This was accomplished by adjusting the laser intensity, detector voltage and detector current so that the three peaks (m/z 5914±12, 7764±16, 9284±19) consistently present in the pooled sera standard were displayed to exact, predetermined criteria. Specifically, the resolution values for all three peaks are required to be greater than 400, and the signal to noise ratios, are ≧40 for m/z 5914, and ≧80 for m/z 7764, and 9284. Two such instruments have been synchronized using these criteria.

A second consideration is the enforcement of a blinded and random (unbiased) sample analysis. To achieve this a grid is drawn; the ProteinChip® used for affinity capture of sera proteins has 8 spots each and is processed 12 chips per unit in a 96 well format. An in-house program was written to assign samples within the grid to prevent bias between triplicate or clinical status and grid position. All samples were processed and the arrayed chips read in a 48-hour period. The samples were assigned grid positions-by an individual blinded to the processing phase and the code was broken during the classification phase. Each sample was processed in triplicate and the values averaged prior to analysis

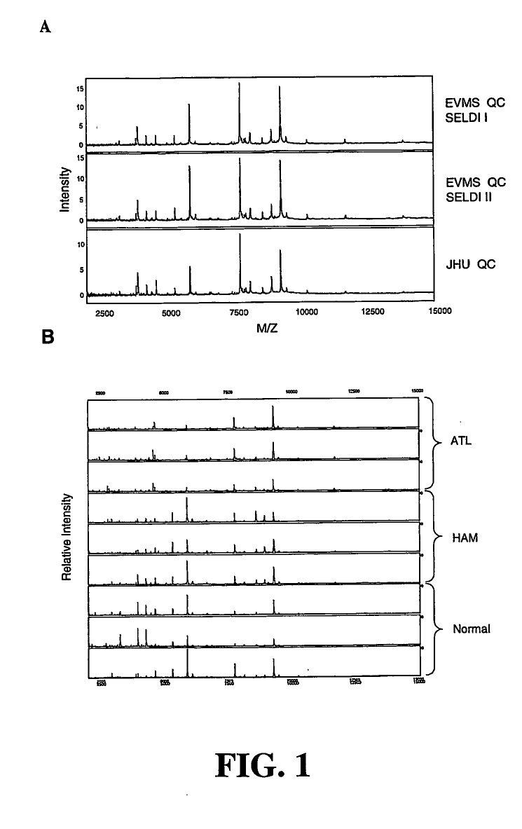

The reproducibility of the spectral data may be seen by referring to FIGS. 1A and 1B. FIG. 1A depicts SELDI profiles of the same serum sample processed on the three different instruments indicated. FIG. 1B depicts spectra from three separate representative individuals from each class. The variation between identical sample spectra was less than 0.2 percent for mass designation and expression amplitude displayed a CV of 15 to 20%.

Peak Mining for Differential Protein Expression Profile

The task of identifying individual peaks that vary from sample to sample and class-to-class is an essential part of any high-throughput mass spectroscopy-based proteomic approach. Prioritization and ranking is achieved based upon either presence or absence in a class and relative expression levels. The Biomarker Wizard utility (Ciphergen Biosystems) was used to prepare the data for classification analysis. After calibration and normalization of the entire data set, consistent peak sets or clusters present in at least 10% of each group are generated based on a mass window of 0.2%. Intensity values are reported for each peak set and differences between groups can be identified. Thus, as mentioned above, peaks are identified based upon being greater or lesser expressed in ATL, HAM or Normal. In addition, peaks are identified as being specifically present in ATL, HAM or control classes.

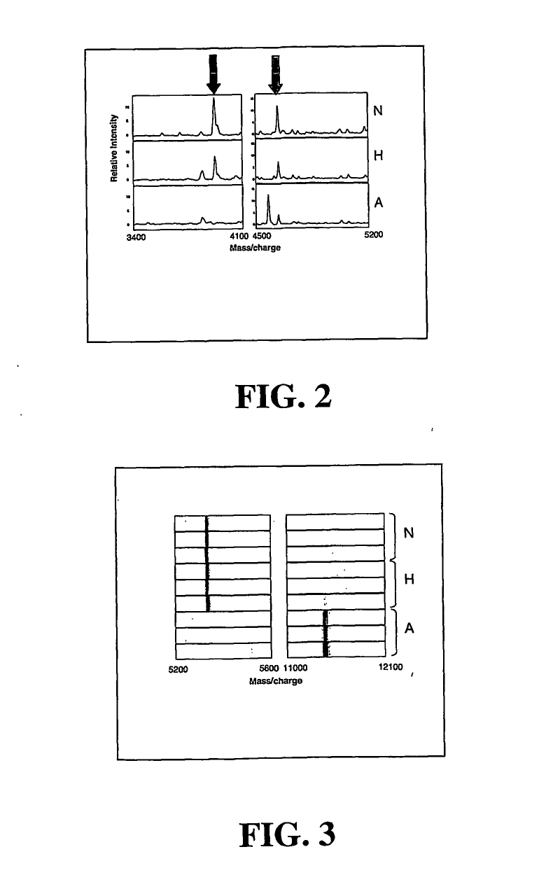

Using this selection process, a number of potential classifier peaks was found for ATL and HAM (63 total peaks). Several types of differential peak events were scored: 1) a peak was over-expressed or under-expressed in a specific class; 2) a peak was progressively expressed from normal to HAM to ATL or the inverse; 3) a peak was only present in a specific class or only missing in a specific class. Examples of these biomarkers are shown in FIGS. 2 and 3 and are discussed herein.

Application of a Decision Tree Algorithm for Classification Value of the Identified Peaks

The verification of the utility of individual peaks as diagnostic biomarkers was addressed using analysis by Classification And Regression Tree (CART). CART analysis is known in the art and described, for example, in Breiman, L., Friedman, J., Olshen, R., and Stone, C. J. (1984) Classification and Regression Trees, Chapman and Hall, New York. Peak values (63 total peaks) determined as described herein were entered and asked for fit-value assignments to each class. The results are shown in Table 2.

TABLE 2
Classification rate of SELDI profiling as determined with
CART. The classes are ATL (A), HAM (H), and Normal (N).

| | | Misclassified | Percent | Classification |
|---|---|---|---|---|
| Study | Class N | N | Error | Rate |
| A vs. N | 15A | 0A | 0 | 100 |
| | 10N | 0N | 0 | 100 |
| H vs. N | 20H | 2H | 10 | 90 |
| | 10N | 2N | 20 | 80 |
| 3-way | 15A | 1A | 6.67 | 93.3 |
| | 20H | 4H | 20 | 80 |
| | 10H | 3N | 30 | 70 |

Referring to Table 2, the ability to distinguish normal from ATL (100% of ATL and 100% of normal correctly identified using 6 peaks representing the protein biomarkers having a molecular weight of about 19900±40, about 11738±23, about 5202±11, about 4577±9, about 4425±8 and about 3965±8 Daltons) or normal from HAM (90% of HAM and 80% of Normal correctly identified using 6 peaks representing the protein biomarkers having a molecular weight of about 11738±23, about 7444±15, about 6144±12, about 4577±9, about 5343±11 and about 9152±18 Daltons) was quite high. In the three-way analysis, individual class identification was reduced from the didactic analysis but still quite high (93.3% of ATL, 80% of HAM and 70% of normal correctly identified using 9 peaks representing the protein biomarkers having a molecular weight of about 19900±40, about 11738±23, about 9094±18, about 7444±15, about 6144±12, about 5914±12, abou 4577±9, about 4425±8, and about 3965±8 Daltons). The CART analysis also revealed the peak values that contained the greatest variable importance. These values are targeted following visual verification for purification purposes.

Each of the peaks in FIGS. 2, 3 and 4 were utilized by the CART approach. Interestingly, when viral load was used as a classifier, none of the disease specific peaks were significantly correlated. Thus, the disease-specific expression profile is likely from the host and not the virus directly.

Application of Cumulative Probability Classification Scheme

ATL 100% correctly classified

HAM 75% correctly classified

NOR 100% correctly classified

Group 2

The experiment described for Group 1 was repeated with a larger sample size similarly acquired and prepared as in Group 1. Study Group 2 consisted of 48 ATL, 60 HAM, and 50 normal controls. The Biomaker Wizard utility discussed above for Group 1 that determines user-defined criteria for which peaks are potentially useful classifiers was utilized in Group 2. Using this selection process, a number of potential classifier peaks were found for ATL and HAM as seen in Table 3.

Table 3 shows selected peaks in Group 2 that are either
differentially expressed, present or absent between groups.
The observed mass (in Daltons) for each of the selected
peaks are shown. The overall prevalence of over-
expressed/under-expressed peaks is given for each and the
class specific fold expression. The mass (in Daltons) of
peaks displaying presence or absence between groups are
listed with the group specific prevalence.
Overexpressed/Underexpressed Peaks

| | Prev. | Fold | Fold | Fold |
|---|---|---|---|---|
| Mass | % | ATL | HAM | NOR |
| 8943 | 100 | 1.8 | 1.8 | 1.0 |
| 11738 | 100 | 2.6 | 1.3 | 1.0 |
| 8609 | 97 | 2.0 | 1.5 | 1.0 |
| 5911 | 87 | −2.5 | 1.0 | 1.0 |
| 4285 | 74 | 2.1 | 2.0 | 1.0 |
| 2793 | 45 | 2.0 | 1.0 | 1.0 |
| 11948 | 32 | 2.3 | 1.0 | 1.0 |
| 19900 | 10 | 4.3 | 2.2 | 1.0 |

Presence/Absence Peaks

| | Presence | Presence | Presence |
|---|---|---|---|
| Mass | in ATL | in HAM | in NOR |
| 6116 | 20% | 60% | 80% |
| 2793 | 63% | 44% | 18% |
| 5343 | 42% | 80% | 97% |
| 2955 | 9% | 35% | 45% |
| 19900 | 14% | 7% | 2% |

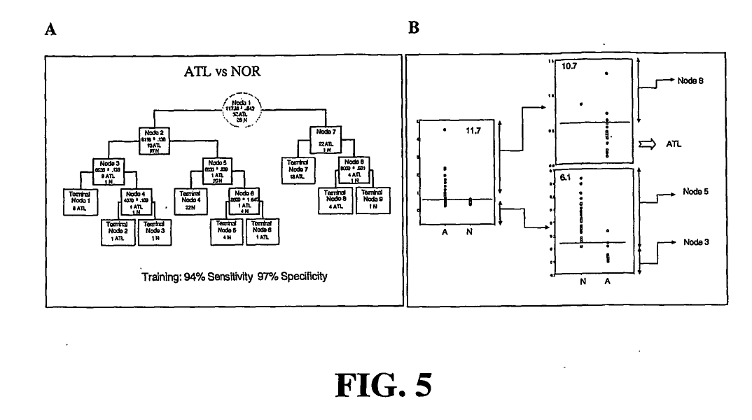

Just as in Group 1, the verification of the utility of individual peaks as diagnostic biomarkers was addressed using analysis by Classification And Regression Tree (CART). The CART software bundle uses a similar ranking process to evaluate peaks for the ability to distinguish between classes and then applies fit-value assignments to each class. A number of potential trees arose from the training and cross-validation and were ranked with respect to classification success. The top performing training trees were subjected to a blinded test set and the final tree selected with the highest classification rate. The algorithm was similary directed to segregate via 3 schemes; ATL vs. Normal; ATL vs. HAM+Normal; and HAM vs. Normal. The tree decisions operate by utilizing simple numeric threshold values for expression of selected peaks. To illustrate this process, the actual relative values in a scatter plot for each splitter peak in the ATL vs. Normal tree are shown in FIG. 5B. In this decision tree, a peak at 11.7 kD was able to distinguish between ATL and normal effectively. However, the best separation in this group was achieved with eight peaks.

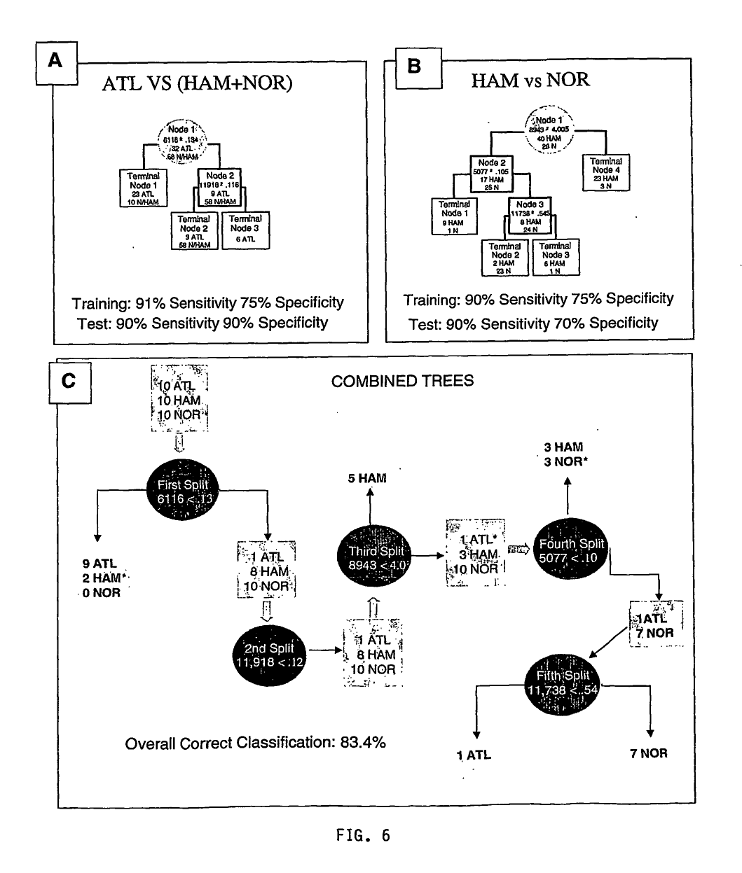

The ability to distinguish ATL from normal was achieved with 94% sensitivity and 97% specificity using a 5 fold cross validation of the training set. The blinded test set resulted in 90% of ATL correctly classified ( 9/10) and 100% of normals correctly classified ( 10/10). Although it is useful to distinguish ATL from non-ATL, the most useful clinical separation is between ATL, HAM and normal. In order to achieve this separation, two didactic trees, ATL vs HAM+normal, and HAM vs. normal, were employed. The application of the regression tree analysis resulted in the trees shown in FIG. 6.

Referring now to FIG. 6A, cross-validation and training for the ATL vs. HAM+normal resulted in 91% sensitivity and 75% specificity. The blinded test set achieved 90% correct classification of ATL ( 9/10) and 90% correct classification of HAM and normal. Likewise, cross validation and training for HAM vs. normal resulted in a sensitivity of 90% and specificity of 75% as seen in FIG. 6B. The results of the blinded test set in this group achieved 90% correct classification of HAM and 70% correct classification of normal as further seen in FIG. 6B. Referring now to the decision structure for the combined trees shown in FIG. 6C, when the two trees were combined and ran in series, 90% correct classification of ATL ( 9/10), 70% correct classification of HAM ( 7/10) and 70% correct classification of normal ( 7/10) was achieved. It should be noted that these results were achieved using a simple classification and regression tree. The simplicity of this design suggests the protein peak profiles that are significant in the ATL group.

As with Group 1, when viral load was used as a classifier, none of the disease specific peaks were significantly correlated. Thus, the disease-specific expression profile is likely from the host and not the virus directly.

### EXAMPLE 2

### Purification and Identification of HTLV Biomarker Peaks

Purification of HTLV Biomarker Peaks

A purification scheme for identifying the SELDI-designated peaks has been developed. The samples that are targeted for isolation and purification are determined by the SELDI profile, which reveals the samples with the greatest differential for expression of the desired protein/peptide. The purification and analysis is applied to the pair so that a comparison is available throughout the purification/identification scheme. Prior to isolating and identifying the biomarkers by liquid chromatograph/mass spectrometry/mass spectrometry (LC/MS/MS), the biomarkers are first isolated by sodium dodecyl sulfate 12% polyacrylamide gel electrophoresis (SDS-PAGE).

For the in-gel trypsin digest, SDS-PAGE gel slices were cut into 1-2 mm cubes, washed 3× with 500 μL Ultra-pure H₂O, and incubated in 100% acetonitrile for 45 minutes. If the gel was silver stained, the stain was first removed with SilverQuest™ destaining solution following manufacturer's instructions. The material was completely dried in a speed-vac and rehydrated in a 12.5 ng/μL modified sequencing grade trypsin solution (Promega) and incubated in an ice bath for approximately 45 minutes. The excess trypsin solution was then removed and replaced with enough 50 mM ammonium bicarbonate, pH 8.0 to cover the gel slice, typically 50 μL. The digest was allowed to proceed overnight at 37° C. Peptides were extracted twice with 25 μL 50% acetonitrile, 5% formic acid and dried in a speed-vac. The peptides were resuspended in 5% acetonitrile, 0.5% formic acid, 0.005% heptafluorobutyric acid (Buffer A), and 3-6 μL applied to a 70 μM ID, 15 cm Magic C18 reverse-phase capillary column. Peptides were eluted with a 5%-80% acetonitrile gradient (Buffer A+95% acetonitrile) and analyzed on a ThermoFinnigan LCQ DECA XP Ion Trap tandem mass spectrometer in positive ion mode. For each scan, the 3 highest intensity ions were subjected to ms/ms analysis. Sequence analysis was performed with Sequest™ using an indexed human subset database of the non-redundant protein database from NCBI.

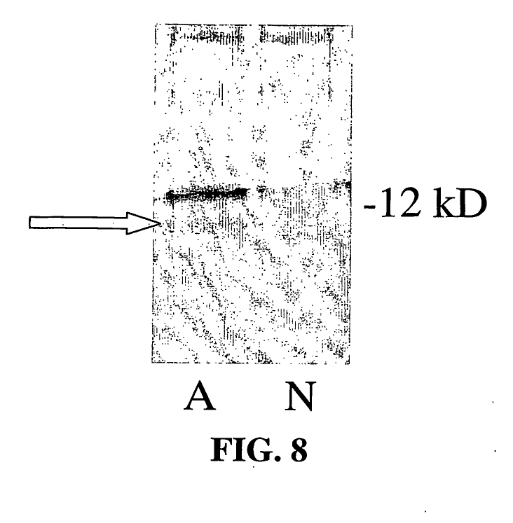

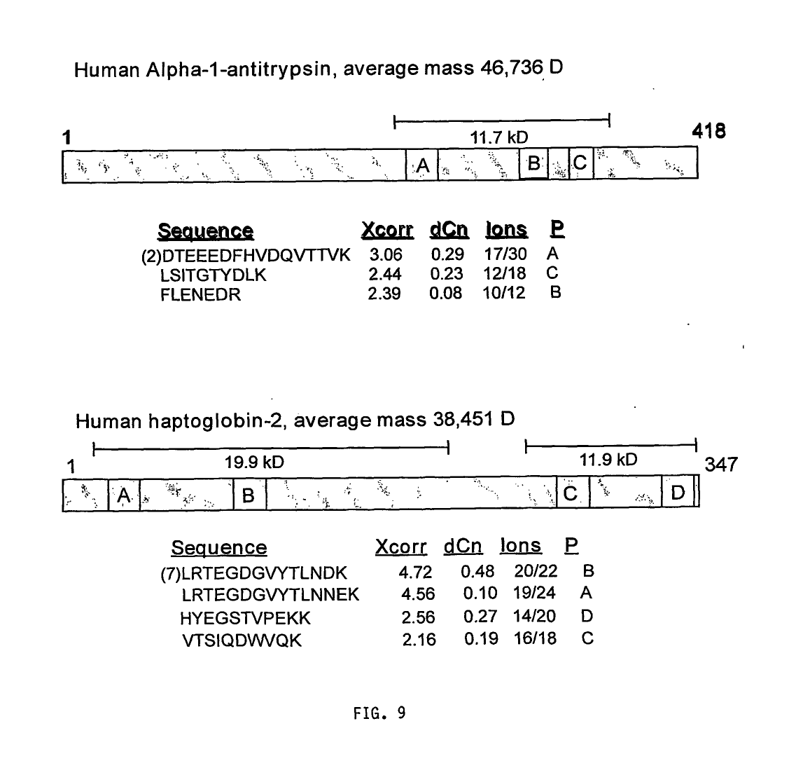

As mentioned above, the purification and analysis is applied to the pair so that a comparison is available throughout the purification/identification scheme. Specifically, after the biomarkers were isolated as described above, the paired samples were first reacted with an off-chip Cu²⁺ affinity column that emulates the on-chip affinity process of the SELDI. This step also greatly reduces serum globulins. The affinity concentrated samples were confirmed on SELDI and then applied to one-dimensional SDS PAGE and silver stained (FIG. 8). The visibly differentially expressed bands within the targeted size range were excised in pairs and analyzed by capillary LC coupled to electrospray tandem mass spectrometry.

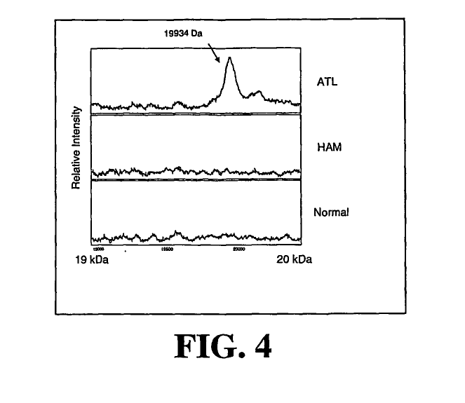

FIG. 4 discussed in Group 1, Example 1, shows the matched SELDI spectra of a 19.9 kD peak specific for ATL. The affinity-eluted fraction was separated by SDS PAGE and visualized with SyproRuby. The specific 20 kD band was excised from the gel. The recovery process was improved by preclearing the sample of imidiazole prior to interaction with the SELDI IMAC3 chip.

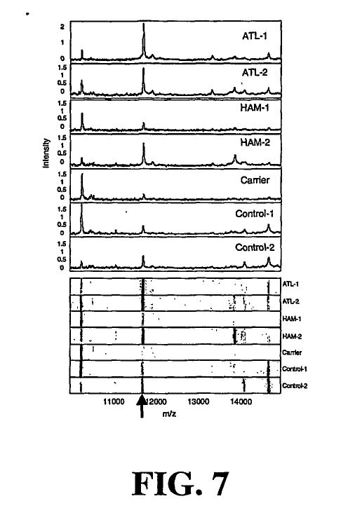

A similar protocol was used to purify an 11.9 kD fragment. Briefly, the ATL and normal serum pairs were reacted with IMAC Cu2+ beads in batch under the same conditions as employed for the SELDI affinity chip surface. The bound proteins were eluted in a single batch wash with reducing PBS (pH=5). The pH was adjusted to 7.0 and the sample loaded onto a 6%/16% gradient gel. The gel was stained with Fast Silver and the bands developed. The region of the stained gel containing the putative 11.7 kD peak (Arrow) is shown in FIG. 7. The band was excised, digested in gel and subjected to LC/MS/MS.

Identification of HTLV Biomarker Peaks

Each of these peptide identities discussed in this section were supported by sequence: coverage consistent with the proposed mass and were excised from bands differentially expressed. SDS-PAGE gel slices were cut into 1-2 mm cubes, washed 3× with 500 μL Ultra-pure H₂O, and incubated in 100% acetonitrile for 45 minutes. If the gel was silver stained, the stain was first removed with SilverQuest™ destaining solution following manufacturer's instructions. The material was completely dried in a speed-vac and rehydrated in a 12.5 ng/μL modified sequencing grade trypsin solution (Promega) and incubated in an ice bath for approximately 45 minutes. The excess trypsin solution was then removed and replaced with enough 50 mM ammonium bicarbonate, pH 8.0 to cover the gel slice, typically 50 μL. The digest was allowed to proceed overnight at 37° C. Peptides were extracted 2× with 25 μL 50% acetonitrile, 5% formic acid and dried in a speed-vac. The peptides were resuspended in 5% acetonitrile, 0.5% formic acid, 0.005% heptafluorobutyric acid (Buffer A), and 3-6 μL applied to a 70 μM ID, 15 cm Magic C18 reverse-phase capillary column. Peptides were eluted with a 5%-80% acetonitrile gradient (Buffer A+95% acetonitrile) and analyzed on a ThermoFinnigan LCQ DECA XP Ion Trap tandem mass spectrometer in positive ion mode. For each scan, the 3 highest intensity ions were subjected to ms/ms analysis. Sequence analysis was performed with Sequest™ using an indexed human subset database of the non-redundant protein database from NCBI.

Using this approach, 19.9 kD and 11.9 kD fragments (i.e., a length or portion of) were identified that represent contiguous halves of haptoglobin-2 (FIG. 9). The sequence of mammalian haptoglobin is set forth in SEQ ID NO:2, and the nucleotide sequence encoding this protein is set forth in SEQ ID NO:1. Interestingly, a unique consensus site for proline protease exists in haptoglobin-2, the cleavage of which would result in the two fragments.

Referring to FIG. 9, peptides A and B of mammalian haptoglobin, were identified by mass spectrometry as explained above as an aid in the identification process, are found in the 19900±40. Peptide A of mammalian haptoglobin (set forth in SEQ ID NO:11) extends from amino acid 60 to amino acid 71 of SEQ ID NO:2. Peptide B of mammalian haptoglobin (set forth in SEQ ID NO:5) extends from amino acid 119 to amino acid 131 of SEQ ID NO:2. The sequence from the amino terminus of peptide A of mammalian haptoglobin to the carboxyl terminus of peptide B extends from amino acid 60 to amino acid 131 of SEQ ID NO:2 and is encoded by the nucleotide sequence set forth in SEQ ID NO:1 from nucleotide 204 to nucleotide 419.

Further referring to FIG. 9, peptides C and D of mammalian haptoglobin, which were also identified by mass spectrometry as explained above as an aid in the identification process, are found in the 11948±24 protein biomarker. Peptide C of mammalian haptoglobin (set forth in SEQ ID NO:6) extends from amino acid 253 to amino acid 263 of SEQ ID NO:2. Peptide D of mammalian haptoglobin (set forth in SEQ ID NO:7) extends from amino acid 333 to amino acid 342 of SEQ ID NO:1. The sequence from the amino terminus of peptide C of mammalian haptoglobin to the carboxyl terminus of peptide D extends from amino acid 253 to amino acid 342 of SEQ ID NO:2 and is encoded by a nucleotide sequence set forth in SEQ ID NO:1 from nucleotide 783 to nucleotide 1052.

The 11.7 kD peak has been identified by the above procedure as a fragment of α-1-anti-trypsin inhibitor, as seen diagrammatically in FIG. 9. The amino acid sequence of mammalian α-1-anti-trypsin inhibitor is set forth in SEQ ID NO:4, and the nucleotide sequence encoding this protein is set forth in SEQ ID NO:3.

Further referring to FIG. 9, peptides A, B and C, which were identified by mass spectrometry as explained above as an aid in the identification process, are found within the 11.7 kD fragment. Peptide A (set forth in SEQ ID NO:8) extends from amino acid 226 to amino acid 241. Peptide B (set forth in SEQ ID NO:9) extends from amino acid 299 to amino acid 305. Peptide C (set forth in SEQ ID NO:10) extends from amino acid 315 to amino acid 324. Therefore, the sequence from the amino terminus of peptide A to the carboxyl terminus of peptide C extends from amino acid 226 to amino acid 324 of SEQ ID NO:4 and is encoded by the nucleotide sequence of mammalian alpha-1-antitrypsin set forth in SEQ ID NO:3 from nucleotide 680 to nucleotide 976.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

What is claimed is:

1. A method for aiding in a diagnosis of HTLV-I-associated myelopathy or adult T cell leukemia, comprising:
(a) detecting at least one protein biomarker in a test sample, said protein biomarker having a molecular weight selected from the group consisting of about 2488±5, about 2793±6, 2955±6, about 3965±8, about 4285±9, about 4425±8, about 4577±9, about 4913±10, about 5202±11, about 5343±11, about 5830±12, about 5874±12, about 5911±12, about 6116±12, about 6144±12, about 6366±13, about 7304±15, about 7444±15, about 8359±17, about 8609±17, about 8943±18, about 9094±18, about 9152±18, about 10113±20, about 11738±23, about 11948±24, about 12480±25, about 14706±29, and about 19900±40 Daltons;
(b) correlating the detection with a probable diagnosis of HTLV-I-associated myelopathy, adult T-cell leukemia or a negative diagnosis.

2. The method of claim 1, wherein said protein biomarkers are selected from the group consisting of about 3965±8, about 4425±8, about 4577±9, about 5343±11, about 8359±17, about 11738±23, and about 19900±40 Daltons.

3. The method of claim 1, wherein said protein biomarker is an 11738±23 Dalton fragment of mammalian alpha-1-antitrypsin.

4. The method of claim 3, wherein said fragment comprises an amino acid sequence set forth in SEQ ID NO:4 from amino acid 226 to amino acid 324.

5. The method of claim 3, wherein said fragment comprises an amino acid sequence encoded by a nucleotide sequence set forth in SEQ ID NO:3 from nucleotide 680 to nucleotide 976.

6. The method of claim 1, wherein said protein biomarker is an 11948±24 Dalton fragment of mammalian haptoglobin

7. The method of claim 6, wherein said fragment comprises an amino acid sequence set forth in SEQ ID NO:2 from amino acid 253 to amino acid 342.

8. The method of claim 6, wherein said fragment comprises an amino acid sequence encoded by a nucleotide sequence set forth in SEQ ID NO:1 from nucleotide 783 to nucleotide 1052.

9. The method of claim 1, wherein said protein biomarker is a 19900±40 Dalton fragment of mammalian haptoglobin.

10. The method of claim 9, wherein said fragment comprises an amino acid sequence set forth in SEQ ID NO:2 from amino acid 60 to amino acid 131.

11. The method of claim 9, wherein said fragment comprises a nucleotide sequence set forth in SEQ ID NO:1 from nucleotide 204 to nucleotide 419.

12. The method of claim 1, wherein said detecting at least one protein biomarker is performed by mass spectroscopy.

13. The method of claim 12, wherein said mass spectroscopy is laser desorption mass spectroscopy.

14. The method of claim 12, wherein said mass spectroscopy is surface enhanced laser desorption/ionization mass spectroscopy.

15. The method of claim 14, wherein the laser desorption/ionization mass spectroscopy includes:
(a) providing a substrate comprising an adsorbent attached thereto;
(b) contacting the test sample with the adsorbent;
(c) desorbing and ionizing the at least one biomarker from the substrate; and
(d) detecting the desorbed/ionized at least one biomarker with a mass spectrometer.

16. The method of claim 15, further comprising purifying the test sample prior to contacting the test sample with the adsorbent.

17. The method of claim 1, wherein said detecting at least one protein biomarker is performed by an immunoassay.

18. The method of claim 17, wherein the immunoassay is an enzyme immunoassay.

19. The method of claim 18, wherein said enzyme immunoassay is an enzyme-linked immunosorbent assay.

20. The method of claim 1, wherein the test sample is blood serum.

21. The method of claim 1, wherein said detecting at least one protein biomarker further comprises identifying the differential expression of said biomarkers.

22. The method of claim 1, wherein one to twenty-nine biomarkers are detected.

23. The method of claim 1, wherein said method comprises:
(a) detecting the presence of protein biomarkers having a molecular weight selected from the group consisting of about 2488±5, about 5202±11, about 7304±15, about 12480±25 and about 19900±40 Daltons, or detecting the absence of protein biomarkers having a molecular weight selected from the group consisting of about 3965±8, 5830±12, 6366±13, 8359±17 and about 9152±18 Daltons; and
(b) correlating the detection with a probable diagnosis of adult T-cell leukemia.

24. The method of claim 1, wherein said method comprises:
(a) detecting the differential expression of protein biomarkers having a molecular weight selected from the group consisting of about 2793±6, about 4285±9, about 4425±8, about 4577±9, about 5343±11, about 5874±12, about 5911±12, about 9094±18, about 10113±20, about 11738±23, about 11948±24, about 13369±27, about 14706±29 and about 19900±40 Daltons; and
(b) correlating the detection with a probable diagnosis of adult T-cell leukemia.

25. The method of claim 1, wherein said method comprises:
(a) detecting the presence or absence of protein biomarkers having a molecular weight selected from the group consisting of about 4913±10, about 6144±12, and about 7444±15 Daltons; and
(b) correlating the detection with a probable diagnosis of HTLV-I-associated myelophathy.

26. The method of claim 1, wherein said method comprises:
(a) detecting the differential expression of protein biomarkers having a molecular weight selected from the group consisting of about 4577±9, about 8613±17, and about 19900±40 Daltons; and
(b) correlating the detection with a probable diagnosis of HTLV-I-associated myelopathy.

27. A method for detecting a protein biomarker in a test sample, comprising detecting the biomarker by a method selected from the group consisting of immunoassay and mass spectrometry, said protein biomarker present, absent or differentially expressed in subjects diagnosed with an HTLV-I-mediated disease selected from the group consisting of HTLV-I-associated myelopathy and adult T-cell leukemia, said protein biomarker having a molecular weight selected from the group consisting of about 2488±5, about 2793±6, 2955±6, about 3965±8, about 4285±9, about 4425±8, about 4577±9, about 4913±10, about 5202±11, about 5343±11, about 5830±12, about 5874±12, about 5911±12, about 6116±12, about 6144±12, about 6366±13, about 7304±15, about 7444±15, about 8359±17, about 8609±17, about 8943±18, about 9094±18, about 9152±18, about 10113±20, about 11738±23, about 11948±24, about 12480±25, about 14706±29, and about 19900±40 Daltons.

28. The method of claim 27, wherein said protein biomarker is an 11738±23 Dalton fragment of mammalian alpha-1-antitrypsin.

29. The method of claim 28, wherein said fragment comprises an amino acid sequence set forth in SEQ ID NO:4 from amino acid 226 to amino acid 324.

30. The method of claim 28, wherein said fragment comprises an amino acid sequence encoded by a nucleotide sequence set forth in SEQ ID NO:3 from nucleotide 680 to nucleotide 976.

31. The method of claim 27, wherein said protein biomarker is an 11948±24 Dalton fragment of mammalian haptoglobin

32. The method of claim 31, wherein said fragment comprises an amino acid sequence set forth in SEQ ID NO:2 from amino acid 253 to amino acid 342.

33. The method of claim 31, wherein said fragment comprises an amino acid sequence encoded by a nucleotide sequence set forth in SEQ ID NO:1 from nucleotide 783 to nucleotide 1052.

34. The method of claim 27, wherein said protein biomarker is a 19900±40 Dalton fragment of mammalian haptoglobin.

35. The method of claim 34, wherein said fragment comprises an amino acid sequence set forth in SEQ ID NO:2 from amino acid 60 to amino acid 131.

36. The method of claim 35, wherein said fragment comprises a nucleotide sequence set forth in SEQ ID NO:1 from nucleotide 204 to nucleotide 419.

37. The method of claim 27, wherein said mass spectrometry is laser desorption/ionization mass spectrometry.

38. The method of claim 27, wherein said immunoassay is an enzyme immunoassay.

39. The method of claim 38, wherein said enzyme immunoassay is an enzyme-linked immunosorbent assay.

40. A kit, comprising:
(a) a substrate comprising an adsorbent attached thereto, wherein the adsorbent is capable of retaining at least one protein biomarker selected from the group consisting of about 2488±5, about 2793±6, 2955±6, about 3965±8, about 4285±9, about 4425±8, about 4577±9, about 4913±10, about 5202±11, about 5343±11, about 5830±12, about 5874±12, about 5911±12, about 6116±12, about 6144±12, about 6366±13, about 7304±15, about 7444±15, about 8359±17, about 8609±17, about 8943±18, about 9094±18, about 9152±18, about 10113±20, about 11738±23, about 11948±24, about 12480±25, about 14706±29, and about 19900±40 Daltons; and
(b) instructions to detect the protein biomarker by contacting a test sample with the adsorbent and detecting the biomarker retained by the adsorbent.

41. The kit of claim 40, wherein the substrate is a probe adapted for use with a gas phase ion spectrometer, said probe having a surface onto which the adsorbent is attached.

42. The kit of claim 40, wherein the adsorbent is a metal chelate adsorbent.

43. The kit of claim 40, wherein the adsorbent comprises a cationic group.

44. The kit of claim 40, wherein the substrate comprises a plurality of different types of adsorbent.

45. The kit of claim 40, wherein the adsorbent comprises an antibody that specifically binds to the biomarker.

46. The kit of claim 40, wherein the kit further comprises (1) an eluant wherein the biomarker is retained on the adsorbent when washed with the eluant.

47. The method of claim 40, wherein said protein biomarker is an 11738±23 Dalton fragment of mammalian alpha-1-antitrypsin.

48. The method of claim 47, wherein said fragment comprises an amino acid sequence set forth in SEQ ID NO:4 from amino acid 226 to amino acid 324.

49. The method of claim 47, wherein said fragment comprises an amino acid sequence encoded by a nucleotide sequence set forth in SEQ ID NO:3 from nucleotide 680 to nucleotide 976.

50. The method of claim 40, wherein said protein biomarker is an 11948±24 Dalton fragment of mammalian haptoglobin

51. The method of claim 50, wherein said fragment comprises an amino acid sequence set forth in SEQ ID NO:2 from amino acid 253 to amino acid 342.

52. The method of claim 50, wherein said fragment comprises an amino acid sequence encoded by a nucleotide sequence set forth in SEQ ID NO:1 from nucleotide 783 to nucleotide 1052.

53. The method of claim 40, wherein said protein biomarker is a 19900±40 Dalton fragment of mammalian haptoglobin.

54. The method of claim 53, wherein said fragment comprises an amino acid sequence set forth in SEQ ID NO:2 from amino acid 60 to amino acid 131.

55. The method of claim 53, wherein said fragment comprises a nucleotide sequence set forth in SEQ ID NO:1 from nucleotide 204 to nucleotide 419.
